(19) United States
(12) Patent Application Publication
(10) Pub. No.: US 2022/0297374 A1
(43) Pub. Date: Sep. 22, 2022

(54) STRUCTURE THAT COMPRESSES OR EXPANDS A VOLUME IN RESPONSE TO AN APPLIED MAGNETIC FIELD

(71) Applicant: Palo Alto Research Center Incorporated, Palo Alto, CA (US)

(72) Inventors: Kathryn F. Murphy, San Francisco, CA (US); Junhua Wei, Mountain View, CA (US); Jerome Unidad, San Francisco, CA (US)

(21) Appl. No.: 17/208,368

(22) Filed: Mar. 22, 2021

(51) Int. Cl. B29C 64/106; B29C 64/209

(52) U.S. Cl. CPC B29C 64/106; B29C 64/209; B33Y 70/00

(57) ABSTRACT

A structure includes a plurality of elongated members flexibly coupled together. The flexible coupling between the elongated members allows the structure to transition between a first and second outer shape. Each of the elongated members has embedded ferromagnetic particles aligned to cause the respective elongated member to move in a predetermined deflection relative to each other in response to an applied magnetic field. The predetermined deflections cause the elongated members to collectively move the structure between the first and second outer shapes. An enclosure is coupled to the structure. The movement of the structure between the first and second shapes performs at least one of compressing and expanding a volume of the enclosure

### SUMMARY

The present disclosure is directed to a structure that compresses or expands a volume in response to an applied magnetic field. In one embodiment, an apparatus comprises a structure with a plurality of elongated members flexibly coupled together. The flexible coupling between the elongated members allows the structure to transition between a first and second outer shape. Each of the elongated members has embedded ferromagnetic particles aligned to cause the respective elongated member to move in a predetermined deflection relative to each other in response to an applied magnetic field. The predetermined deflections cause the elongated members to collectively move the structure between the first and second outer shapes. An enclosure is coupled to the structure. The movement of the structure between the first and second shapes performs at least one of compressing and expanding a volume of the enclosure.

In another embodiment, a method involves locating a deformable structure in a target location. The deformable structure includes a plurality of elongated members flexibly coupled together. The flexible coupling between the elongated members allows the structure to transition between a first and second outer shape. Each of the elongated members includes embedded magnetic particles aligned to cause the respective elongated member to move in a predetermined deflection relative to the each other in response to a magnetic field. The method involves applying the magnetic field to the structure in the target location to cause the elongated members to collectively move the deformable structure between the first and second outer shapes. The movement of the structure between the first and second shapes compressing or expanding a volume of an enclosure coupled to the structure.

In another embodiment, a method involves placing feedstock into a dispensing unit. The feedstock includes magnetic particles and a flowable material. A flow of the feedstock is caused in a flow direction out of an orifice of the dispensing unit and towards a build surface. A field is applied onto the fluid flow that causes an alignment of the magnetic particles. At least one of the build surface and the orifice are moved such that the fluid flow exiting the orifice additively manufactures a structure, the structure comprising a plurality of elongated members flexibly coupled together. The flexible coupling between the elongated members allows the structure to transition between a first and second outer shape. The field is varied over time such that the magnetic particles are aligned in the elongated members to cause the respective elongated member to move in a predetermined deflection relative to the each other in response to an applied magnetic field. The predetermined deflections cause the elongated members to collectively move the structure between the first and second outer shapes. These and other features and aspects of various embodiments may be understood in view of the following detailed discussion and accompanying drawings.

### BRIEF DESCRIPTION OF THE DRAWINGS

The discussion below makes reference to the following figures, wherein the same reference number may be used to identify the similar/same component in multiple figures.

FIG. 1 is a diagram of a two-dimensional structure according to an example embodiment;

FIG. 2 is a perspective view of a structure used to compress or expand a cuboid volume according to an example embodiment;

FIG. 3 is a perspective view of a structure used to compress or expand a cylindrical volume according to an example embodiment;

FIGS. 4 and 5 are a plan and perspective views of a structure formed using Hoberman mechanisms according to an example embodiment;

FIG. 6 is a perspective view of a structure used to compress or expand a toroidal volume according to an example embodiment;

FIG. 7 is a perspective view of a structure with a helicoid shape according to an example embodiment;

FIG. 8 is a perspective view of a structure used to compress or expand a paraboloid or similar volume according to an example embodiment;

FIG. 9 is a perspective view of a structure used to compress or expand a hyperboloid volume according to an example embodiment;

FIG. 10 is a diagram of an apparatus that can be used to manufacture structure according to the various embodiments; and

FIGS. 11 and 12 are flowcharts of methods according to example embodiments.

### DETAILED DESCRIPTION

The present disclosure is generally related to mechanical actuators. In fields such as robotics, manufacturing, automation, etc., many different types of actuators are used. Generally, an actuator will take energy as an input, and produce motion as an output. The input energy could be mechanical, electrical, hydraulic, pneumatic, etc., and the output motion could be linear movement, rotation, clamping, separation, impacts, etc. The present disclosure relates to actuator applications in which it is desired to induce actuation without requiring a physical link (e.g., wires, fluid lines) to the actuator in order to induce the actuation.

One way of performing remote actuation without a physical link is to equip the actuation device with its own power supply, and then use a radio transmission to actuate it. While this may work in some applications, in other applications a power supply such as a battery may not be acceptable. For example, for devices that are implanted in living organisms, batteries may contain chemicals that are harmful to the organisms.

In this disclosure, methods and apparatuses are described that can actuate an object remotely upon trigger, with no tethers or internal power sources. One type of actuation that can be performed by these apparatuses involves applying pressure to an object, e.g., to squeeze a sac or balloon containing a drug or beneficial micro-organisms for controlled dispensing inside a body cavity or body part. The same concept could work for other applications which require squeezing, such as inducing constrictions around body parts, grabbing objects (e.g., for soft robots), pumping liquids, pumping gases, etc.

In one embodiment, an apparatus has a plurality of elongated members flexibly coupled together into a structure. The flexible coupling between the elongated members allows the structure to transition between a first outer shape and a second outer shape. Each of the elongated members include embedded magnetic particles aligned to cause the respective elongated member to move in a predetermined deflection relative to the each other in response to an applied magnetic field. The predetermined deflections cause the elongated members to collectively move the structure between the first and second outer shapes.

Generally, the various embodiments described herein do not require any of an internal power source, port, tether, wire, tube, or other material object that extends outside the body. These embodiments may be formed with materials that are partially or entirely biocompatible or biodegradable. Compared to other forms of artificial muscles (e.g. electroactive polymers), large strains (or volume changes) can be achieved without requiring large voltages and the actuation can be induced without tethers to the actuator. The geometry of the embodiments can also be adapted to various geometric requirements depending on the squeezing or volume change required.

The embodiments described herein (compared to other competing approaches) are robust to mechanical failure of the materials used in the apparatus. For example, a failure in one segment of the apparatus will not necessarily result in drastic loss of function of the rest of the apparatus. Additive manufacturing techniques (e.g., 3-D printing) are also described that can form a wide variety of different shapes as well as ensure predefined shape changes in response to an applied magnetic field, function,

A specific use for the apparatus is for an implantable drug delivery device to be located in the gastric cavity or in other parts of the gastrointenstinal tract. In this particular application, the apparatus is filled with a payload of active ingredients (e.g. drugs, biologics or cells) that need to be dispensed internally within a desired schedule. This dispensing will be triggered externally with the application of a field that causes the squeezing or volume change of the apparatus that expels a determined amount of the payload into the cavity. It is envisioned that such an apparatus will remain inside the body for some duration, e.g. until all the drug payload has been expelled, such that repeat triggering of the squeezing or volume change according to a schedule will be performed.

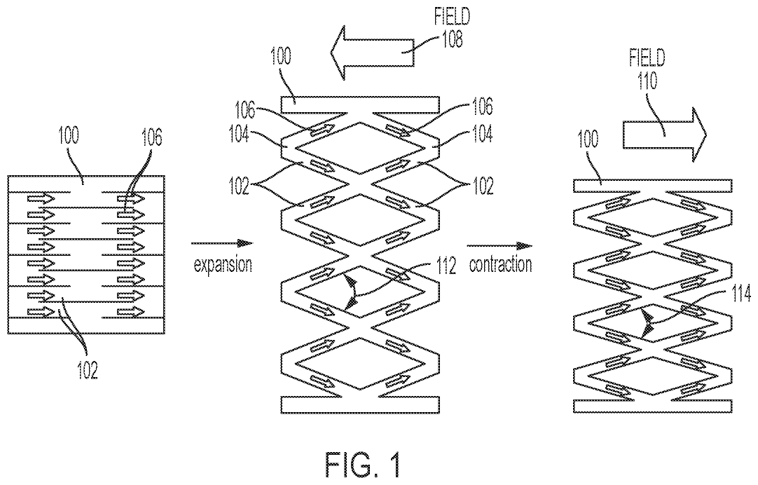

In FIG. 1, a diagram shows details of a remotely actuated structure 100 that can collapse and/or expand in response to an external magnetic field according to an example embodiment. The structure 100 includes a plurality of ligaments 102 (also referred to herein as elongated members) flexibly coupled at joints 104. The ligaments 102 are filled with ferromagnetic particles which have magnetic orientations 106. The particles are oriented such that, when external magnetic fields 108, 110 are applied, the ligaments 102 deflect so their magnetic orientations 106 are aligned with the external field 108.

The device shown in FIG. 1 resembles a kirigami paper cutting, in which 2D sheets of paper are cut so that the paper can be deformed into a variety of shapes. In the left side of FIG. 1, the structure 100 is shown in an initial state resembling a sheet that is cut so that it can expand to multiples of its original length. Instead of cutting a sheet of material, an expandable lattice structure such as this can be 3D printed with magnetic particles aligned so that upon application of a magnetic field, the structure will expand or contract, resulting in compression.

As seen in the middle of FIG. 1, a first applied field 108 opposite to the magnetic field orientations 106 causes the compact sheet to expand into a first shape. This results in the ligaments 102 crossing one another at a first angle 112 in the first shape. As seen in the right side of FIG. 1, when a second field is applied 110, the lattice ligaments align along the field causing the structure 100 to compress. This results in the ligaments 102 crossing each other at a second angle 114 in the second shape.

For purposes of this disclosure, the lattice arrangement seen in this structure 100 will be referred to as a “scissors mechanism,” as its deflections involve elongated members rotating relative to one another about joints, where the elongated members and joints form a repeated cross or diamond pattern. The example in FIG. 1 is a two-dimensional sheet so the applied fields 108, 110 only need lie along one dimension, and the magnetic orientations 106 can be the same over all of the ligaments 102. Also note that the deflected outer shapes in the middle and right hand sides of FIG. 1 may not remain entirely two-dimensional, as the ligaments 102 and or joints 104 may bend out of plane in order to maintain the illustrated deflections.

In some embodiments, the joints 104 may rely on elastic deformation of the material from which the structure 100 is made, e.g., a flexible polymer. The additive manufacturing of the structure 100 can add features to ensure the bending is predictable, such as changing a material composition between the joints 104 and ligaments 102, changing a size, cross-section, or other geometry of the joints 104 and ligaments to increase or decrease relative stiffness, etc. Also, the filaments added to obtain the magnetic field orientations 106 may increase stiffness of the ligaments 102, and this material can be left out of the joints. In other embodiments, the joints 104 may be made from known mechanical constructs, such as hinges, ball and sockets, shaft and bushing, etc. In the latter case, the structure itself need not be made from flexible material, as the mechanical construction of the joint can provide the desired flexibility of the structure.

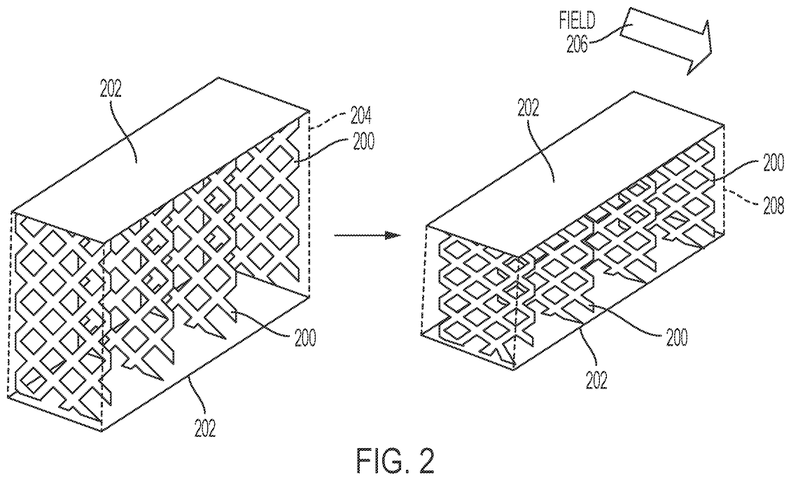

In some applications, the structure 100 can be used to compress a volume. While some embodiments described herein extend similar structures into three-dimensional shapes, a two-dimensional shape as shown in FIG. 1 can be used to compress a three-dimensional volume. An example of this is shown in FIG. 2, which is a perspective view of an apparatus according to an example embodiment. In this example, four expandable structures 200 are shown between top and bottom membranes 202. The membranes 202 have rectangular first shapes that define a cuboid volume 204. The cuboid volume 204 is compressed by the structures 200, and other membranes may enclose the volume 204, including front and back membranes and side membranes. In some embodiments, more or fewer structures 200 may be used. For example, a bellows-shaped volume may operate with single structure 200.

The structures 200 elongated members having embedded magnetic particles aligned to cause the respective elongated member to move in a predetermined deflection relative to the each other in response to an applied magnetic field 206, as seen in the right hand side of FIG. 1. In this example, the application of the field 206 causes the structures 200 to compress to a second shape (a smaller rectangle), causing the membranes 202 to enclose a cuboid volume 208 which is smaller than cuboid volume 204.

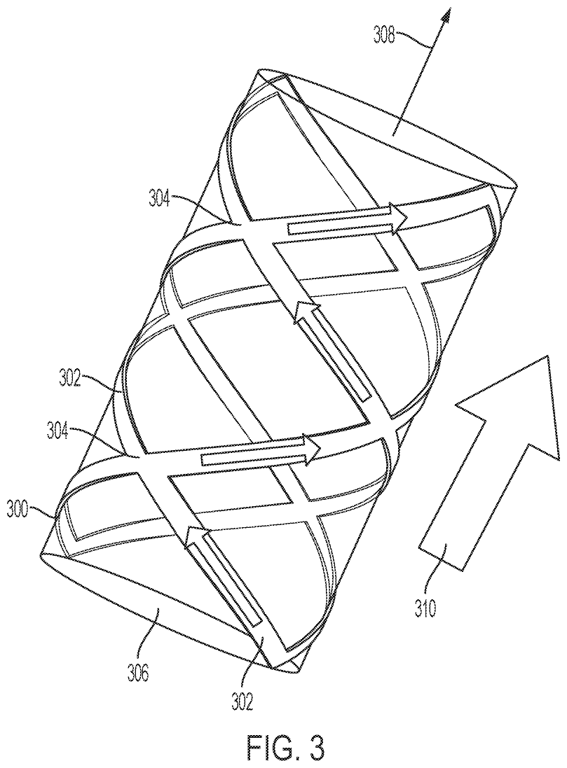

In other embodiments, a structure such as shown in FIG. 1 can be wrapped or folded in three-dimensions, allowing a scissors-type mesh to enclose a volume, with or without additional enclosure membranes. In FIG. 3, a perspective view shows a sheet structure wrapped into a cylinder structure 300 according to an example embodiment. The cylinder structure 300 includes a plurality of elongated members 302 flexibly coupled at joints 304. The elongated members 302 are in a scissors-type meshed that is wrapped around a cylindrical volume 306. In response to an applied field 310, the cylinder structure can compress or expand along a longitudinal axis 308, forming first and second cylindrical shapes. An apparatus may use the structure 300 as part of an enclosure, e.g., adding sealing membranes along the sides and ends. Compression of the structure 300 can dispense a liquid and/or gas through an orifice upon compression, for example. In another example, the structure 300 can be repeatedly compressed and expanded such that, in combination with other component such as a one-way valve, the structure 300 can be part of a pumping apparatus.

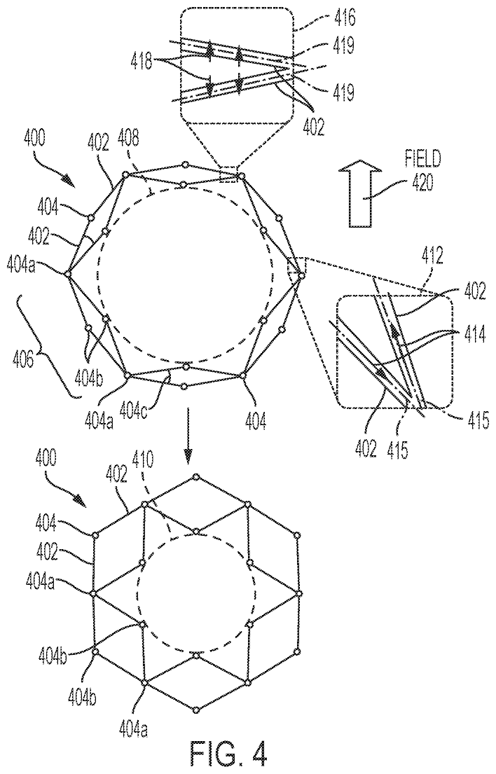

In FIG. 4, a diagram shows a mechanism 400 that may be used to form structures according to other example embodiments. This mechanism 400 is referred to as a Hoberman mechanism or Hoberman linkage. The mechanism 400 includes a plurality of elongated members 402 flexibly coupled at joints 404. The elongated members 402 may include construction as described for other embodiments, including flexibly deformable joints 404 and/or mechanisms used to form the joints, structural and/or material differences between joints 404 and members 402, etc.

The elongated members 402 are arranged in groups of four, e.g., group 406, with outside joints 404a that link the group 406 to other similar groups. In this example, six groups 406 are linked together to form the mechanism 400, although more or fewer groups 406 may be used. Inside joints 404b allow the group 406 of elongated members 402 to increase or decrease their angles 404c at the outside joints 404a, which simultaneously and inversely changes the distance between the two inside joints 404b and the distance between the outside joints 404a.

The groups 406 are linked together in circular shape that encompasses a circular area 408. The mechanism 400 can expand to the extent that opposing inside joints 404b of the same group 406 contact one another. The mechanism 400 can contract to the extent that two opposing inside joints 404b from different groups 406 facing one another come into contact. In the lower part of FIG. 4, the mechanism 400 is shown contracted relative to the view at the top of the figure, resulting in the mechanism encompassing a circular area 410 that is smaller than circular area 408.

In order to remotely cause expansion and contraction of the mechanism 400, the elongated members 402 are embedded with magnetic particles that may be aligned during manufacture to respond appropriately to an applied field 420. As shown in detail view 412, this may include alignments 414 that are approximately parallel to the major axes 415 of the elongated members 402. However, a similar alignment elsewhere on the mechanism 400, e.g., at a 90° offset to the location of view 412, may not have the desired effect. In one embodiment, only a subset of the groups 406 may have magnetic particles so as to avoid counteracting forces in some conditions. Or, as seen in detail view 416, other regions may have different alignments 418 relative to the major axes of the elongated members 402, in this case approximately normal to the major axes 419. Magnetic orientations may range continuously between different relative angles throughout the mechanism 400 in order to achieve the desired effect.

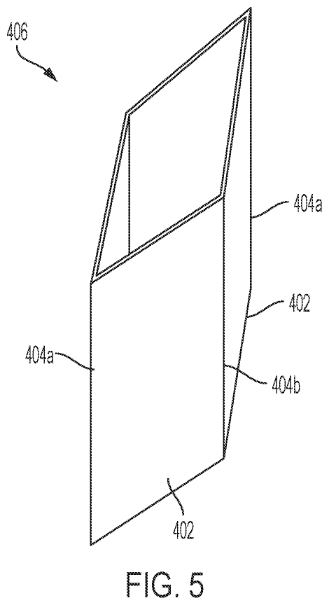

The mechanism 400 can be extruded out of the plane of the page to create a three-dimensional structure that can compress or expand an internal volume. An example of a group 406 that is expanded this way is shown in the perspective view of FIG. 5. A number of these groups 406 can be used to form an approximation of a cylinder when linked together into a circular perimeter similar to mechanism 400 shown in FIG. 4. In order to reduce the amount of material used for high aspect ratio cylinders (large ratio of height to radius), a number of mechanisms 400 can be stacked upon each other and joined together by elongated linking members.

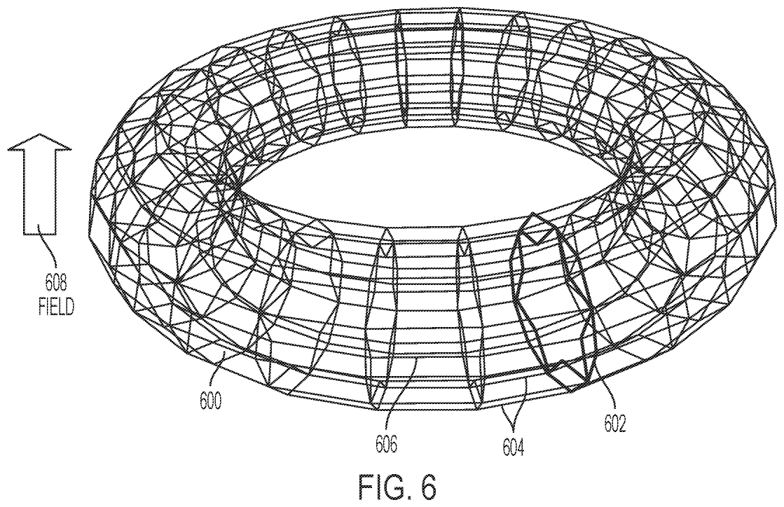

Linking of Hoberman mechanisms 400 can be used to form other three-dimensional shapes. In FIG. 6, a perspective view shows a toroidal structure 600 according to an example embodiment. This structure 600 is formed by joining a plurality of Hoberman mechanisms 602 by linking members 604 into a toroidal shape. An applied field 608 will cause the structure 600 to compress or expand a toroidal-shaped inner volume 606. In a compressed state, the inner diameter of the structure 600 may remain much the same due to tensile strength of linking members 604 on or near the inner diameter being greater than buckling resistance of the linking members 604 on or near the outer diameter. The Hoberman mechanisms 602 can be formed with location-dependent magnetic material orientations as describe in relation to FIG. 4.

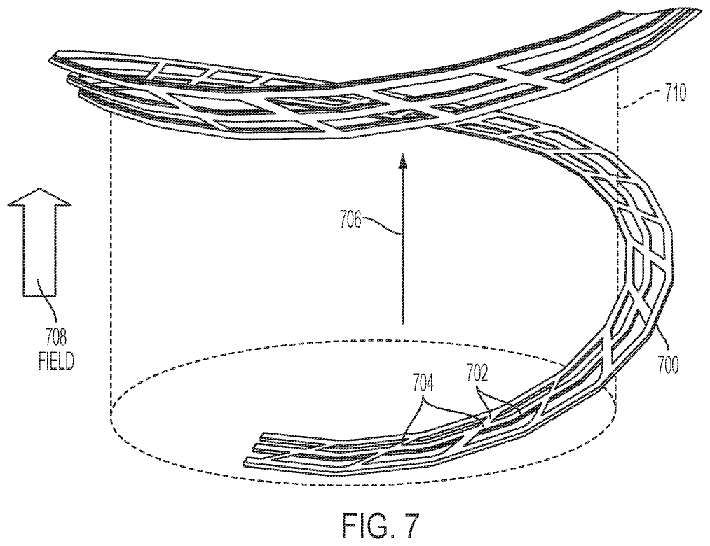

In FIG. 7, a perspective view shows a helicoid structure 700 according to an example embodiment. The structure 700 includes a plurality of elongated members 702 flexibly coupled at joints 704 using a scissors-mechanism grid. The mesh of scissors mechanisms could be more extensive than shown, and may extend continuously from an outer diameter to a central axis 706. Depending on a magnetic orientation of the particles within the elongated members 702, the structure 700 could be cause to compress and expand along the central axis 706 in response to an applied field 708, thereby compressing a cylindrically shaped inner volume 710. In some embodiments, the structure 700 could be configured to radially constrict the inner volume 710 in response to the field 708.

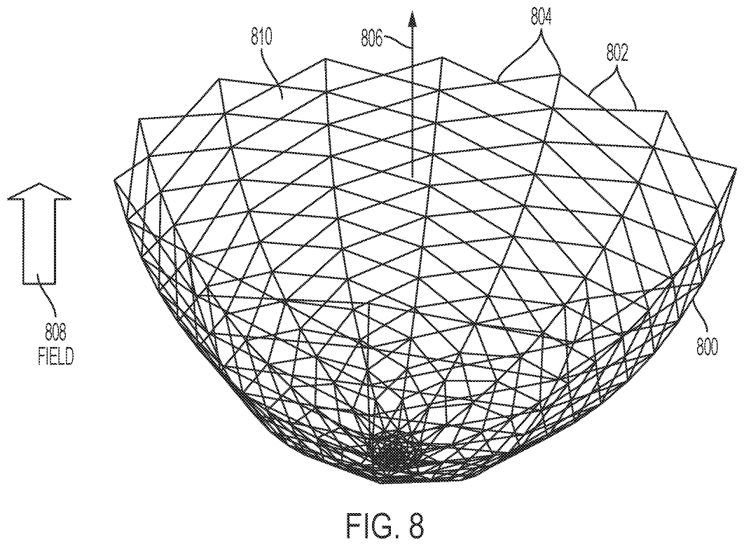

In FIG. 8, a perspective view shows a paraboloid structure 800 according to an example embodiment. The structure 800 includes a plurality of elongated members 802 flexibly coupled at joints 804 in a scissors-mechanism grid. The magnetic orientation of particles within the elongated members 802 can be arranged so that the structure 800 compresses and expands towards a central axis 806 in response to an applied field 808, thereby compressing a paraboloid-shaped inner volume 810. Note that construction of this structure can be slightly modified to form other three-dimensional shapes, including a cone, spherical sections, and ellipsoidal sections. Further, two mirror images of such shapes can be joined together to form closed shapes such as spheres, spheroids, ellipsoids, and can be combined with other shapes, such as capsule shape (spherocylinder) which has two spherical ends joined by a cylider.

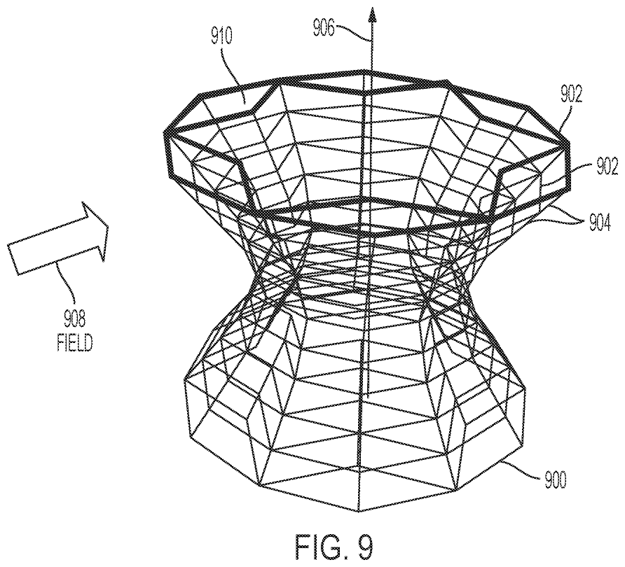

Also note that structures similar to structure 800 may be formed using Hoberman mechanisms. For example, Hoberman mechanisms 400 can be formed as seen in FIG. 4 with different maximum outer diameters. A stack of such Hoberman mechanisms can be connected via linking members (e.g., members 604 shown in FIG. 6; also see linking members 904 between stacked Hoberman mechanisms 902 in FIG. 9). The outer dimensions of the mechanisms 400 and length of linking members can be tailored to form any of the shapes described above. The Hoberman mechanisms 400 could be arranged to compress/expand the resulting structure towards a central axis, normal to the central axis, or both, e.g., as in a Hoberman sphere.

In FIG. 9, a perspective view shows a hyperboloid structure 900 according to an example embodiment. The structure 900 includes a plurality of Hoberman mechanisms 902 of different maximum outer diameters that are stacked upon one another and coupled via linking members 904. The outer dimensions of the Hoberman mechanisms 902 and length of linking members 904 can be selected to achieve the hyperboloid outer shape of the structure 900, which also encloses a hyperboloid inner volume 910. The magnetic orientation of particles within the elongated members of the Hoberman mechanisms 902 can be arranged so that the structure 900 compresses and expands towards a central axis 906 in response to an applied field 908, thereby compressing a hyperboloid inner volume 910. In another embodiment, adjacent stacked Hoberman mechanism 902 (or another circular shaped structure) could be linked by a scissors mesh instead of the vertical linking members 904, allowing the structure to compress/expand in the direction of the central axis 906 instead of (or in addition to) moving toward/away from the axis 906.

Any of the embodiments described above may be formed in an additive manufacturing process. Generally, in these processes, external fields are used to orient portions of a composite material that is used in an extruded additive manufacturing process. This technology enables composite systems with the ability to control the composites properties in three dimensions at all points within a single part. The process works by taking advantage of a large change in viscosity during the printing process. The embedded particles are first oriented in the lower viscosity portion of the process by a three-dimensional external field. The particles are then locked into places as the material hardens (either by curing or cooling) and viscosity rises.

The objects formed using these processes may be composites of polymers (or other flowable materials) and other materials using feedstocks comprising polymer and powders of materials with anisotropic magnetic properties. The polymer forms the matrix and one or more other components are oriented within the polymer matrix using external fields to give enhanced properties. The method allows for voxel-level control of field-specific properties (e.g., magnetization, polarization) in the printed part. Note that the flowable material need not be a polymer. The method may also apply to other materials such as glass, plant and animal waxes, etc.

In one embodiment, a mixture of a flowable material (e.g., a polymer) and orientable/functionally-anisotropic materials (referred to herein as “functional materials” and include magnetic particles) is used to create parts with on-demand patterning of the field-specific property within each voxel. For example, the feedstocks can have permanent, internal magnetic or electric fields where the orientation of the field can be controlled in three dimensions at the voxel level during printing. The polymer component is mixed with the filler and extruded in the presence of an external field to create a permanent orientation in the solid part. In other cases, the orientation of functional materials can, instead of or in addition to orienting magnetic fields, result in other directionally oriented properties, such as structural strength and heat transfer (e.g., conductivity).

The polymer used as flowable material for the structures described herein is preferentially a curable elastomer, including but not limited to one or more of silicone, polyisoprene, polybutadiene, chloroprene, polychloroprene, neoprene, fluorosilicone, perfluoroelastomer, polyether block amides, chlorosulfonated polyethylene, or ethylene-vinyl acetate. The polymer may also comprise biodegradable polymers or hydrogels including polysaccharides, proteins, or biopolyesters; or thermoplastic elastomers including styrenic block copolymers, polyolefinelastomers, vulcanizates, polyurethanes, copolyesters, or polyamides.

The feedstock can be a mixture of the flowable material and magnetic material plus other components, for example surfactants/compatibilizers to help with adhesion, materials to help with the polymer flow, or particles which will tune mechanical properties (e.g. clay, silica, carbon nanotubes, graphene). The feedstock may be a granulate composite, with the ferromagnetic material being encapsulated by a polymer or other flowable materials. An example of such composite feedstocks could be samarium cobalt permanent magnet (SmCo) core with polypropylene (PP) coating. The feedstock could instead or in addition include separate functional material and flowable material mixed together.

Note that embodiments described herein may utilize any shape and proportion of the feedstock components. For example, the functional and flowable materials may be configured as fibers, shards, etc. The flowable material may be a solid at the working temperature of the resulting device, e.g., room temperature, and is heated to allow it to flow together with the functional material, where it again hardens and fixes the orientation of the functional material. In other embodiments, the flowable material uses chemical reactions to assist or cause hardening. For example, the flowable material may be in a liquid state at the working temperature (e.g., room temperature) and thereby does not require melting, and may be cured by the application of heat and/or light after being deposited. The functional materials may include ferrous and non-ferrous metals, dielectrics, carbon fiber, graphene, etc. Further, the feedstock may be provided in other forms, such as block, filament, liquid solution, etc.

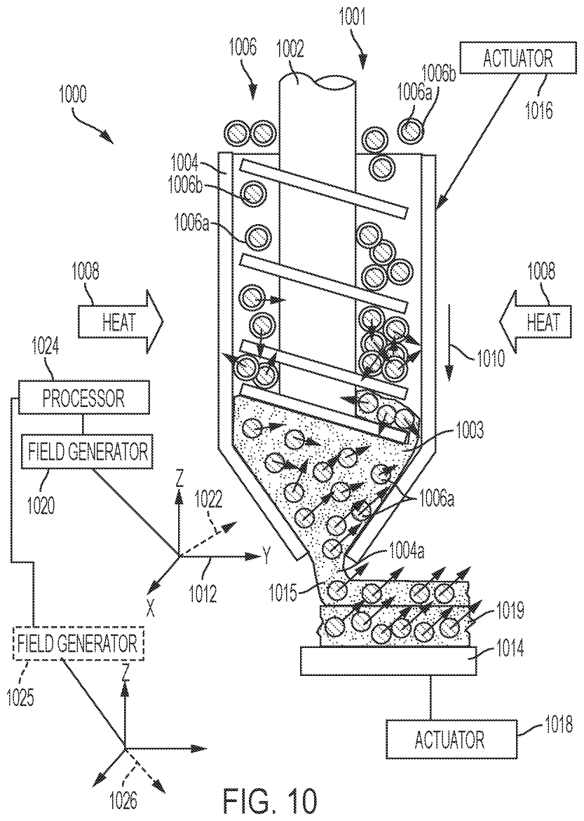

In FIG. 10 a schematic diagram illustrates an extrusion system 1000 according to an example embodiment. The extrusion system 1000 includes a manufacturing apparatus with a feeding mechanism 1001 that moves feedstock 1006 into a dispensing unit 1004. In this example, the feeding mechanism 1002 is a feed screw housed within a nozzle, the nozzle acting as the dispensing unit 1004. Feedstock 1006 is fed into the feed screw where it heated to form a fluid flow 1003 that is directed in a flow direction 1010 (which corresponds to the z-axis in the illustrated coordinate system 1012) towards an orifice 1004a of the dispensing unit 1004 The feedstock 1006 includes both a functional material 1006a and a flowable material 1006b as previously described.

A heat source, indicated by arrows 1008, may optionally be used to heat the feedstock 1006 which is at least partially melted to create a uniform mixture within the fluid flow 1003, which carries the functional material 1006a within. Note that as described above, the flowable material may be liquid at the working temperature (e.g., room temperature) such that melting is not needed. The fluid flow 1003 exits the orifice 1004a where it is deposited onto a build surface 1014, indicated here as region of deposition 1015. One or both of the dispensing unit 1004 and build surface 1014 can be moved relative to one another, e.g., via actuators 1016, 1018.

The material deposited on the build surface 1014 (or previously deposited feedstock on the build surface 1014) will cool and solidify, rapidly increasing in viscosity and locking the particles 1006a into their intended orientation, which is set while in a liquid state via a field generator. Additional cooling can be provided depending on the ratio of viscosity to field strength for the composite materials, e.g., via fans, chemical reactions of the flowable material when in contact with ambient air. A part 1019 can be formed by successive passes of the flow 1003 in a predetermined pattern. The part 1019 is formed of a polymer matrix of the solidified functional material 1006a and flowable material 1006b.

The movement of the actuators 1016, 1018 together with the extrusion of material from the dispensing unit 1004 facilitates additively forming the part 1019 on the surface 1014. Note that the relative movement of the dispensing unit 1004 and building surface 1014 via actuators 1016, 1018 can be purely translational (e.g., three degrees of freedom) or any combination of rotational and translational (e.g., up to six degrees of freedom). The build surface 1014 may be planar as shown, or other shapes, e.g., a rotating cylinder that facilitates depositing cylindrical shapes.

One or more field generators 1020 are located proximate to the region of deposition, e.g., near the orifice 1004a of the dispensing unit 1004 where it affects the material flow before leaving the orifice 1004a. The field generator 1020 may be outside of the dispensing unit 1004 or within the dispensing unit 1004. In the latter case, the field generator 1020 may be in contact with the flow 1003. The field generator 1020 generates a field 1022 that can be oriented in three dimensions, which varies the orientation of the functional material 1006b as it passes by.

The field 1022 can be, for example, electric or magnetic fields that align the polarized particles and/or induce internal polarization in the materials. The field 1022 may be configured to have at least one component normal/perpendicular to the flow direction 1010, e.g., a vector that represents the field 1022 having either a positive or negative component on the xy-plane. In the case of SmCo these field generators could be electromagnets which serve to either magnetize the particles (if the feedstock is unmagnetized) or rotate the particles into alignment via the magnetic field (if the feedstock is already magnetized).

As indicated by second field generator 1025, a second field 1026 could also be applied to the flow 1003 in combination with the first field 1022. The second field 1026 could be of a different type (e.g., electrical, magnetic) than the first field 1022. As seen in the figure, the fields 1022, 1026 could have different orientations at any given instant of time. The fields 1022, 1026 could operate on a single type of functional material 1006a, for example enhancing the orientation thereof. In other cases, multiple types of functional material 1006a may be used, each being affected differently by the different fields 1022. Note that any functions ascribed herein to the field generator 1022 may be equally applied to the second field generator 1026.

As indicated by the arrows on the particles of functional materials 1006a within the heated area of the dispensing unit 1004, the functional material particles 1006a transition from a disordered/random alignment to being aligned by the field 1022 as the particles 1006a are deposited onto the building surface 1014. The field generator 1020 is configured by a processor 1024 that changes an angle, direction and/or magnitude of the field 1022 as a function of time. Note that direction and angle of the field 1022 can be interdependent. For example, a 180 degree change in angle will have the same result as changing the direction of the field vector 1022 between positive and negative, which can be accomplished in some embodiments by changing a direction of current in the field generator 1022.

The changes in the field 1022 applied by the processor 1024 are coordinated with the change in relative orientation between the nozzle 1004 and building surface 1014 via the processor 1024 (which is also directly or indirectly coupled to the actuators 1016, 1018) facilitating selectively variable orientation of the functional materials 1006a within a volume of the part 1019. For more details on this manufacturing process and manufacturing apparatuses, reference is made to U.S. patent application Ser. 16/236,852, filed Dec. 31, 2018, which is hereby incorporated by reference.

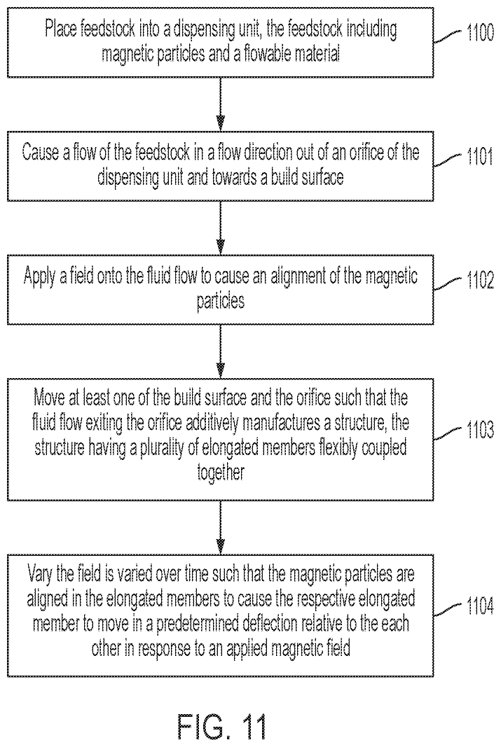

In FIG. 11, a flowchart shows a method according to another example embodiment. The method comprising placing 1100 feedstock into a dispensing unit, the feedstock comprising magnetic particles and a flowable material. A flow of the feedstock is caused 1101 in a flow direction out of an orifice of the dispensing unit and towards a build surface. A field is applied 1102 onto the fluid flow that causes an alignment of the magnetic particles. At least one of the build surface and the orifice are moved 1103 such that the fluid flow exiting the orifice additively manufactures a structure, the structure comprising a plurality of elongated members flexibly coupled together. The flexible coupling between the elongated members allows the structure to transition between a first and second outer shape. The field is varied 1104 over time such that the magnetic particles are aligned in the elongated members to cause, after manufacture, the respective elongated member to move in a predetermined deflection relative to the each other in response to an applied magnetic field. The predetermined deflections cause the elongated members to collectively move the structure between the first and second outer shapes.

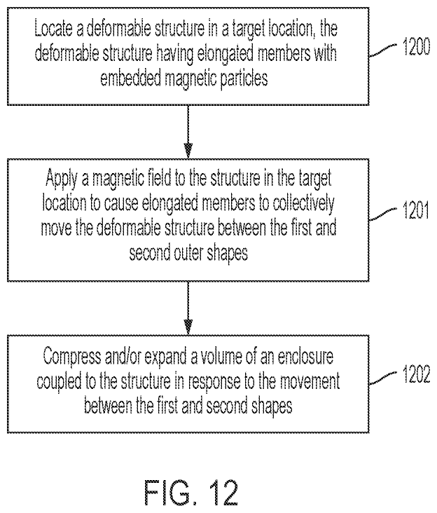

In FIG. 12, a flowchart shows a method according to another example embodiment. The method involves locating 1200 a deformable structure in a target location, such as inside a living organism. The deformable structure includes a plurality of elongated members flexibly coupled together. The flexible coupling between the elongated members allows the structure to transition between a first and second outer shape. Each of the elongated members has embedded magnetic particles aligned to cause the respective elongated member to move in a predetermined deflection relative to the each other in response to a magnetic field.

A magnetic field is applied 1201 to the structure in the target location to cause the elongated members to collectively move the deformable structure between the first and second outer shapes. An enclosure is coupled to the structure, and the movement of the structure between the first and second shapes compresses and/or expands 1202 a volume of the enclosure.

The various embodiments described above may be implemented using circuitry, firmware, and/or software modules that interact to provide particular results. One of skill in the arts can readily implement such described functionality, either at a modular level or as a whole, using knowledge generally known in the art. For example, the flowcharts and control diagrams illustrated herein may be used to create computer-readable instructions/code for execution by a processor. Such instructions may be stored on a non-transitory computer-readable medium and transferred to the processor for execution as is known in the art. The structures and procedures shown above are only a representative example of embodiments that can be used to provide the functions described hereinabove.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g. 1 to 5 includes 1, 1.5, 2, 2.75, 3, 3.80, 4, and 5) and any range within that range.

The foregoing description of the example embodiments has been presented for the purposes of illustration and description. It is not intended to be exhaustive or to limit the embodiments to the precise form disclosed. Many modifications and variations are possible in light of the above teaching. Any or all features of the disclosed embodiments can be applied individually or in any combination are not meant to be limiting, but purely illustrative. It is intended that the scope of the invention be limited not with this detailed description, but rather determined by the claims appended hereto.

1. An apparatus comprising:
a structure comprising a plurality of elongated members flexibly coupled together, the flexible coupling between the elongated members allowing the structure to transition between a first and second outer shape, each of the elongated members comprising embedded ferromagnetic particles aligned to cause the respective elongated member to move in a predetermined deflection relative to each other in response to an applied magnetic field, the predetermined deflections causing the elongated members to collectively move the structure between the first and second outer shapes; and
an enclosure coupled to the structure, the movement of the structure between the first and second shapes performs at least one of compressing and expanding a volume of the enclosure.

2. The apparatus of claim 1, wherein the structure comprises a lattice formed of the elongated members crossing one another at a first angle in the first shape and a second angle in the second shape.

3. The apparatus of claim 2, wherein the structure comprises a two-dimensional sheet wrapped around a three-dimensional inner volume.

4. The apparatus of claim 2, wherein the structure comprises a three-dimensional structure.

5. The apparatus of claim 4, wherein the three-dimensional structure comprises at least one of a cylinder, spheroid, cuboid, ellipsoid, helicoid, cone, paraboloid, hyperboloid, toroid.

6. The apparatus of claim 1, wherein the compressing of the volume within the structure dispenses a gas or liquid from the enclosure.

7. The apparatus of claim 6, wherein the apparatus is fully biocompatible or biodegradable.

8. The apparatus of claim 6, wherein the apparatus is partially biocompatible or biodegradable.

9. The apparatus of claim 6, wherein the structure and enclosure comprise a single-use dispenser.

10. The apparatus of claim 6, wherein the structure and enclosure form a pump, and wherein the applied magnetic field repeatedly compresses and expanding a volume of the enclosure.

11. The apparatus of claim 1, wherein the elongated members are formed of a polymer.

12. The apparatus of claim 11, wherein the embedded ferromagnetic particles are aligned in the elongated members via a directionally-variable magnetic field applied while the polymer is in a flowable state during an additive manufacturing process.

13. The apparatus of claim 11, wherein the polymer comprises a curable elastomer.

14. The apparatus of claim 11, wherein the polymer comprises a biodegradable polymer or hydrogel.

15. A method comprising:
locating a deformable structure in a target location, the deformable structure comprising a plurality of elongated members flexibly coupled together, the flexible coupling between the elongated members allowing the structure to transition between a first and second outer shape, each of the elongated members comprising embedded magnetic particles aligned to cause the respective elongated member to move in a predetermined deflection relative to the each other in response to a magnetic field; and
applying the magnetic field to the structure in the target location to cause the elongated members to collectively move the deformable structure between the first and second outer shapes, the movement of the structure between the first and second shapes compressing or expanding a volume of an enclosure coupled to the structure.

16. The method of claim 15, wherein the structure comprises a lattice formed of the elongated members crossing one another at a first angle in the first shape and a second angle in the second shape.

17. The method of claim 15, wherein the compressing of the volume within the structure dispenses a gas or liquid from the enclosure.

18. The method of claim 17, wherein the target location comprises within a living organism, and wherein the deformable structure is biocompatible or biodegradable.

19. The method of claim 17, wherein the deformable structure and enclosure comprise a pump, and wherein applying the magnetic field comprises varying a direction of the magnetic field to repeatedly compress and expand the volume.

20. A method comprising:
placing feedstock into a dispensing unit, the feedstock comprising magnetic particles and a flowable material;
causing a flow of the feedstock is in a flow direction out of an orifice of the dispensing unit and towards a build surface;
applying a field is onto the fluid flow that causes an alignment of the magnetic particles;
moving at least one of the build surface and the orifice such that the fluid flow exiting the orifice additively manufactures a structure, the structure comprising a plurality of elongated members flexibly coupled together, the flexible coupling between the elongated members allowing the structure to transition between a first and second outer shape;
varying the field is varied over time such that the magnetic particles are aligned in the elongated members to cause, after manufacture, the respective elongated member to move in a predetermined deflection relative to the each other in response to an applied magnetic field, the predetermined deflections causing the elongated members to collectively move the structure between the first and second outer shapes.
